(19) United States
(12) Patent Application Publication
(10) Pub. No.: US 2024/0091282 A1
(43) Pub. Date: Mar. 21, 2024

(54) Probiotic Microcapsule for the Treatment of Reproductive Tract Infection and Its Preparation Method and Application

(71) Applicants: Tangyi Biomedical (Tianjin) Co., Ltd., Tianjin (CN); Tangyi Holding (Shenzhen) Co., Ltd., Shenzhen (CN)

(72) Inventors: Bin ZHENG, Tianjin (CN); Qinglu GUO, Tianjin (CN); Yulin CAO, Beijing (CN); Shixiang CHENG, Tianjin (CN); Zhihui BAI, Beijing (CN)

(73) Assignees: Tangyi Biomedical (Tianjin) Co., Ltd., Tianjin (CN); Tangyi Holding (Shenzhen) Co., Ltd., Shenzhen (CN)

(21) Appl. No.: 17/967,918

(22) Filed: Oct. 18, 2022

(30) Foreign Application Priority Data
Sep. 16, 2022 (CN) 202211134085.3

(51) Int. Cl. A61K 35/747; A61K 9/50; A61K 31/137; A61P 31/04

(52) U.S. Cl. CPC A61K 35/747; A61K 9/5015; A61K 9/5063; A61K 31/137; A61P 31/04

(57) ABSTRACT

A probiotic microcapsule for the treatment of reproductive tract infection and a preparation method and application thereof are provided. The probiotic microcapsule is obtained by coating probiotics with the coating material. The probiotics include one or several of Lactobacillus acidophilus, Lactobacillus rhamnosus, Lactobacillus fermentum, Lactobacillus reuteri, Lactobacillus crimpus, Lactobacillus brevis, Lactobacillus salivary, Lactobacillus plantarum, Lactobacillus acidophilus, and Lactobacillus bulgaricus. As probiotics screened in reproductive tract, it has high safety; It endows probiotics with adhesion function to ensure their effect in the reproductive tract; It overcomes the defects of current antibiotic therapy; It is expected to be used in a variety of reproductive tract infectious diseases.

### CROSS REFERENCE TO THE RELATED APPLICATIONS

This application is based upon and claims priority to Chinese Patent Application No. 202211134085.3, filed on Sep. 16, 2022, the entire contents of which are incorporated herein by reference.

### TECHNICAL FIELD

The invention belongs to the technical field of microbiology, in particular relates to a probiotic microcapsule for the treatment of reproductive tract infection and its preparation method and application thereof.

### BACKGROUND

The level of female reproductive health directly affects the quality of social population, which cannot be ignored. Among them, female reproductive tract infection has the characteristics of high prevalence, low consultation rate and high asymptomatic proportion, which leads to a variety of serious complications and sequelae. Female reproductive tract infection has become one of the most important infectious diseases in the world. Bacterial vaginosis is one of the major diseases of reproductive tract infection. There is still no research on its specific pathogenesis, and the current clinical treatment is mainly the application of antibiotics. However, various antibiotic drugs have many side effects, such as destroying the protective probiotic flora in the vagina and damage to the microecological environment in the reproductive tract, thereby increasing the incidence of recurrent vaginal infections and other reproductive tract infections. Long-term antibiotic treatment also increases the resistance of reproductive tract pathogens and may lead to the development of superbugs.

### SUMMARY

In view of this, the invention aims to provide a probiotic microcapsule for the treatment of reproductive tract infection and a preparation method and application thereof. The probiotic microcapsule provided by the invention is safe for female vagina and can treat female reproductive tract infection.

In order to achieve the purpose of the invention, the invention provides the following technical scheme:
The invention provides a probiotic microcapsule for the treatment of reproductive tract infection. The probiotic microcapsule is obtained by coating probiotics with the coating material;
The probiotics include one or several of Lactobacillus acidophilus, Lactobacillus rhamnosus, Lactobacillus fermentum, Lactobacillus reuteri, Lactobacillus crimpus, Lactobacillus brevis, Lactobacillus salivary, Lactobacillus plantarum, Lactobacillus acidophilus and Lactobacillus bulgaricus.

Preferably, the coating material includes dopamine and/or mussels.

The invention also provides a preparation method of probiotic microcapsules described in the technical scheme, including the following steps:
1) The probiotics were obtained by centrifuging the liquid of the probiotics in the logarithmic growth phase;
2) The probiotics obtained in step 1) were mixed with buffer and coating material to obtain the mixture;
3) The mixture obtained in step 2) was vortexed and centrifuged, and the resulting precipitate was mixed with sterile water to obtain probiotic microcapsules.

Preferably, the number of probiotics in the bacterial solution as described in step 1) is 1×10²−1×10¹⁰ cfu/ml.

Preferably, the buffer in step 2) includes Tris-HCl or PBS solution, and the pH value of the buffer is 5-8;

The ratio of the volume of bacteria solution in step 1) to the volume of buffer solution and the mass of coating material is 1 ml:1 ml:2 mg.

Preferably, the vortexing conditions described in step 3) include: first vortexing for 1-10 min, then vortexing for 1-10 min every 10-100 min, and centrifugation after 10-1000 min.

Preferably, the volume ratio of the bacterial solution in step 1) to the sterile water in step 3) is 1:0.01-100, and the sterile water is sterile ultrapure water.

The invention also provides the application of probiotic microcapsules described in the technical scheme in the preparation of drugs for the treatment of reproductive tract infections.

Preferably, the reproductive tract infection includes bacterial vaginitis.

In the present invention, the probiotics in the probiotics microcapsule achieve the purpose of treating female reproductive tract infection by regulating female reproductive tract microorganisms.

Beneficial effects of the invention are as follows:
(1) As probiotics screened in reproductive tract, it has high safety;
(2) Endow probiotics with adhesion function to ensure their effect in the reproductive tract;
(3) Overcome the defects of current antibiotic therapy;
(4) It is expected to be used in a variety of reproductive tract infectious diseases.

### BRIEF DESCRIPTION OF THE DRAWINGS

FIG. 1 shows probiotics;

FIG. 2 shows dopamine-coated probiotics.

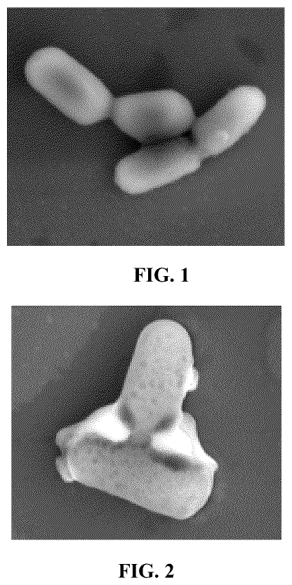

### DETAILED DESCRIPTION OF THE EMBODIMENTS

The invention provides a probiotic microcapsule for the treatment of reproductive tract infection. The probiotic microcapsule is obtained by coating probiotics with the coating material; The probiotics include one or several of Lactobacillus acidophilus, Lactobacillus rhamnosus, Lactobacillus fermentum, Lactobacillus reuteri, Lactobacillus crimpus, Lactobacillus brevis, Lactobacillus salivary, Lactobacillus plantarum, Lactobacillus acidophilus and Lactobacillus bulgaricus. The invention has no special limitation on the source of the probiotics, and it is preferred to come from the female reproductive tract.

In the present invention, the number of probiotics per milliliter of probiotics capsule is preferably 1×10²−1×10¹⁰ cfu/ml, and it is better to be 1×10⁷ CFU. In the present invention, the coating material preferably includes dopamine and/or mussels. In the present invention, the coating material endows probiotics with adhesion ability, and then exerts precise therapeutic effect at the site of infection.

In the present invention, the use method of the probiotic microcapsule preferably includes: 1 ml of probiotic microcapsule is administered vaginally.

The invention also provides a preparation method of probiotic microcapsules described in the technical scheme, including the following steps:
1) The probiotics were obtained by centrifuging the bacterial solution of the probiotics;
2) The probiotics obtained in step 1) were mixed with buffer and coating material to obtain the mixture;
3) The mixture obtained in step 2) was vortexed and centrifuged, and the resulting precipitate was mixed with sterile water to obtain probiotic microcapsules.

In the invention, the probiotics were obtained by centrifuging the bacterial solution of the probiotics. The invention has no special limitation on the centrifugal conditions, and the technical personnel in this field can operate according to the routine operation. The invention has no special limitation on the cultivation method of the bacterial liquid of the probiotics, and the technical personnel in this field can rely on the conventional method of cultivation of the bacteria. The number of probiotics in the bacterial solution is preferably 1×10⁷ cfu/ml.

In the invention, the probiotics obtained were mixed with buffer and coating material to obtain the mixture. In the present invention, the buffer preferably includes Tris-HCl or PBS solution, and the pH value of the buffer is preferably 5-8 and it is better to be 6.8. In the present invention, the ratio of the volume of the bacterial solution to the volume of the buffer solution and the mass of the coating material is preferably 1 ml:1 ml:2 mg.

In the invention, the mixture obtained was vortexed and centrifuged, and the resulting precipitate was mixed with sterile water to obtain probiotic microcapsules. In the present invention, the optimal conditions of the vortex include: first vortexing for 1-10 min, then vortexing for 1-10 min every 10-100 min, and centrifugation after 10-1000 min. In the present invention, the volume ratio of the bacterial solution to the sterile water is preferably 1:0.01-100, and it is better to be 1:1. The sterile water is preferably sterile ultrapure water.

The invention also provides the application of probiotic microcapsules described in the technical scheme in the preparation of drugs for the treatment of reproductive tract infections. In the present invention, the reproductive tract infection includes bacterial vaginitis.

The technical solutions provided by the present invention are described in detail below in conjunction with embodiments, but they shall not be construed as limiting the scope of protection of the present invention.

### Embodiment 1

The bacterial solutions of Lactobacillus acidophilus, Lactobacillus rhamnosus, Lactobacillus fermentum and Lactobacillus Reuteri were centrifuged for 1 mL each, and the bacterial content in the solution was 1*10⁷ CFU/mL. Discard the supernatant, mix each bacterium and add 1 ml of Tris-HCl solution pH=6.8; add 2 mg dopamine, and vortex for 5 min, every 0.5 h for 5 min. After 2 h, centrifuge the supernatant and add 1 ml sterile ultrapure water to obtain probiotic capsule solution.

### Embodiment 2

To verify its efficacy in animal experiments, 1 ml of the microcapsule solution of Embodiment 1 was taken for vaginal administration. Once a day for seven days. Microbial sequencing analysis and vaginal secretion smear observation were used to evaluate the effect of probiotics. Microbial sequencing results showed that the vaginal microbiota returned to normal and no obvious pathogenic bacteria were found in the smear of secretions, indicating that vaginitis had improved.

The foregoing is only a preferred embodiment of the present invention. It should be noted that for ordinary technicians in the technical field, a number of improvements and refinements can be made without deviating from the principle of the invention. These improvements and finishes shall also be considered as the scope of protection of the present invention.

What is claimed is:

1. A probiotic microcapsule for treating a reproductive tract infection, wherein the probiotic microcapsule is obtained by coating probiotics with a coating material;
the probiotics comprises at least one of Lactobacillus acidophilus, Lactobacillus rhamnosus, Lactobacillus fermentum, Lactobacillus reuteri, Lactobacillus crimpus, Lactobacillus brevis, Lactobacillus salivary, Lactobacillus plantarum, Lactobacillus acidophilus, and Lactobacillus bulgaricus.

2. The probiotic microcapsule according to claim 1, wherein the coating material comprises dopamine and/or mussels.

3. A preparation method of the probiotic microcapsule according to claim 1, comprising the following steps:
1) obtaining the probiotics by centrifuging a liquid of the probiotics in a logarithmic growth phase;
2) mixing the probiotics obtained in step 1) with a buffer and the coating material to obtain a mixture;
3) vortexing and centrifuging the mixture obtained in step 2) to obtain a resulting precipitate, and mixing the resulting precipitate with sterile water to obtain the probiotic microcapsule.

4. The preparation method according to claim 3, wherein a number of the probiotics in a bacterial solution as described in step 1) is 1×10²−1×10¹⁰ cfu/ml.

5. The preparation method according to claim 3, wherein the buffer in step 2) comprises Tris-HCl or PBS solution, and a pH value of the buffer is 5-8;
a ratio of a volume of a bacteria solution in step 2) to a volume of a buffer solution and a mass of the coating material is 1 ml:1 ml:2 mg.

6. The preparation method according to claim 3, wherein conditions of the vortexing described in step 3) comprise: first vortexing for 1-10 min and then vortexing for 1-10 min every 10-100 min, and the centrifuging is performed after 10-1000 min.

7. The preparation method according to claim 3, wherein a volume ratio of a bacterial solution in step 1) to the sterile water in step 3) is 1:0.01-100, and the sterile water is sterile ultrapure water.

8. A method of an application of the probiotic microcapsule according to claim 1 in a preparation of drugs for a treatment of the reproductive tract infection.

9. The preparation method according to claim 3, wherein the coating material comprises dopamine and/or mussels.

10. The method according to claim 8, wherein the coating material comprises dopamine and/or mussels.
